(19) United States
(12) Patent Application Publication
(10) Pub. No.: US 2026/0360509 A1
(43) Pub. Date: Nov. 12, 2026

(54) MEAL PLAN GENERATING METHOD, APPARATUS, AND COMPUTER IMPLEMENTED ALGORITHM THEREOF

(71) Applicant: N.V. Nutricia, Zoetermeer (NL)

(72) Inventors: Bingzhi Guo, Shanghai (CN); Mengjin Liu, Shanghai (CN); Mingliang Wang, Shanghai (CN); Yi Jin, Shanghai (CN); Qiwen Song, Shanghai (CN); Lubei Dong, Shanghai (CN)

(73) Assignee: N.V. Nutricia, Zoetermeer (NL)

(21) Appl. No.: 19/719,313

(22) Filed: Jun. 25, 2026

Related U.S. Application Data
(63) Continuation of application No. PCT/CN2023/143199, filed on Dec. 29, 2023.

(51) Int. Cl. G16H 20/60; G16H 10/60

(52) U.S. Cl. CPC G16H 20/60; G16H 10/60

(57) ABSTRACT

The present invention provides a method for generating a meal plan. The method may comprise: obtaining diet preference information of a user; obtaining existing ingredient information; determining a first set of dishes from a predetermined dish set based on the existing ingredient information and the diet preference information of the user; obtaining user information; determining a target value for each intake category based on the user information; obtaining at least one general rule; determining, in the first set of dishes, an actual value for the each intake category; determining a second set of dishes from the predetermined dish set based on differences between the target values and the actual values in the first set of dishes and the at least one general rule; and generating a meal plan comprising the first set of dishes and the second set of dishes.

### FIELD OF THE INVENTION

The present invention relates to a meal plan generating method, apparatus, and computer implemented algorithm thereof.

### BACKGROUND

Nutrition is a critical driver of health and well-being of a person, which are normally obtained by the person from intakes of food (including drinks). Specially, for certain group of people, the nutrition intakes are very critical, e.g., for pregnant or lactating females, people with nutrition related diseases (e.g., diabetes), and specific segments such as: senior/old people having special nutrition intake needs, etc. Therefore, it is very important to control/personalize the nutrition intakes according to the different situation of each targeted person.

Some people plan meals for every day mainly replying on their diet habits and diet preferences, without paying much attention to the balance of nutrition and the total energy intake. As a result, the diversity of food is limited, or people get much more energy than they need, which can result in malnutrition or obesity, or other nutrition-related issues.

Thus, a system providing intelligent diet plans is needed to help people to plan their daily meals.

To ensure that people can obtain a suitable amount of all necessary nutrition and follow the recommended meal plans easily, the present invention provides a meal plan generating method, apparatus, and computer implemented algorithm thereof, which aims to optimize and personalize nutrition intakes and at the same time considers the preference of the users, the ingredients the users have, history of diseases, special stage of the users, for example, pre-pregnancy, pregnancy, puerperium, or lactation, and active level of the users. The targeted user may be a pregnant female, a pregnant female having diabetes, a male, a baby, a child, a senior person, or any other persons.

### SUMMARY OF THE INVENTION

In order to solve the above technical problems, the present invention provides a meal plan generating method, apparatus, and computer implemented algorithm/method thereof.

The present invention aims at providing personalized meal plans for users.

The present invention is according to the claims.

### BRIEF DESCRIPTION OF THE DRAWINGS

The present invention will be discussed in more detail below, with reference to the attached drawings, in which:

FIG. 1 shows a meal plan generating method.

FIG. 2 shows a method of categorising the first set of dishes after the determining of the first set of dishes from the predetermined dish set.

FIG. 3 shows a method of determining the second set of dishes.

FIG. 4 shows a detailed method of determining the extra meal menu.

FIG. 5 shows a detailed method of determining the breakfast menu.

FIG. 6 shows a detailed method of determining the lunch menu.

FIG. 7 shows a detailed method of determining the dinner menu.

FIG. 8 shows an apparatus configured to perform a part of or all steps in the present invention.

### DESCRIPTION OF EMBODIMENTS

Embodiments of the present disclosure will be described herein below with reference to the accompanying drawings. However, the embodiments of the present disclosure are not limited to the specific embodiments and should be construed as including all modifications, changes, equivalent devices and methods, and/or alternative embodiments of the present disclosure.

The terms “have,” “may have,” “include,” and “may include” as used herein indicate the presence of corresponding features (for example, elements such as numerical values, functions, operations, or parts), and do not preclude the presence of additional features.

The terms “A or B,” “at least one of A or/and B,” or “one or more of A or/and B” as used herein include all possible combinations of items enumerated with them. For example, “A or B,” “at least one of A and B,” or “at least one of A or B” means (1) including at least one A, (2) including at least one B, or (3) including both at least one A and at least one B.

The terms such as “first” and “second” as used herein may modify various elements regardless of an order and/or importance of the corresponding elements, and do not limit the corresponding elements. These terms may be used for the purpose of distinguishing one element from another element. For example, a first printing form and a second printing form may indicate different printing forms regardless of the order or importance. For example, a first element may be referred to as a second element without departing from the scope the present invention, and similarly, a second element may be referred to as a first element.

It will be understood that, when an element (for example, a first element) is “(operatively or communicatively) coupled with/to” or “connected to” another element (for example, a second element), the element may be directly coupled with/to another element, and there may be an intervening element (for example, a third element) between the element and another element. To the contrary, it will be understood that, when an element (for example, a first element) is “directly coupled with/to” or “directly connected to” another element (for example, a second element), there is no intervening element (for example, a third element) between the element and another element.

The expression “configured to (or set to)” as used herein may be used interchangeably with “suitable for,” “having the capacity to,” “designed to,” “adapted to,” “made to,” or “capable of” according to a context. The term “configured to (set to)” does not necessarily mean “specifically designed to” in a hardware level. Instead, the expression “apparatus configured to . . . ” may mean that the apparatus is “capable of . . . ” along with other devices or parts in a certain context.

The terms used in describing the various embodiments of the present disclosure are for the purpose of describing particular embodiments and are not intended to limit the present disclosure. As used herein, the singular forms are intended to include the plural forms as well, unless the context clearly indicates otherwise. All of the terms used herein including technical or scientific terms have the same meanings as those generally understood by an ordinary skilled person in the related art unless they are defined otherwise. The terms defined in a generally used dictionary should be interpreted as having the same or similar meanings as the contextual meanings of the relevant technology and should not be interpreted as having ideal or exaggerated meanings unless they are clearly defined herein. According to circumstances, even the terms defined in this disclosure should not be interpreted as excluding the embodiments of the present disclosure.

The present invention provides a method for generating a meal plan. The method may comprise: obtaining diet preference information of a user; obtaining existing ingredient information; determining a first set of dishes from a predetermined dish set based on the existing ingredient information and the diet preference information of the user; obtaining user information; determining a target value for each intake category based on the user information; obtaining at least one general rule; determining, in the first set of dishes, an actual value for the each intake category; determining a second set of dishes from the predetermined dish set based on differences between the target values and the actual values in the first set of dishes and the at least one general rule; and generating a meal plan comprising the first set of dishes and the second set of dishes.

The diet preference information may comprise at least one of taste preference, seasoning preference, cooking type preference, ingredients preference, and cuisine preference, wherein the diet preference information is pre-stored or is obtained from a input module comprising a voice recognition module and/or a text recognition module.

The existing ingredient information may comprise information of one or more ingredients, and the existing ingredient information may be obtained via text input of the user, or recognized from an image, or selection of the user from a predetermined database.

Each of the first set of dishes may comprise at least one of the one, two or more ingredients comprised in the existing ingredient information.

The predetermined dish set may comprise a set of dishes stored in a predetermined database, and the predetermined database may further comprise information on at least one food category and predetermined dish packages. Each of the dish packages may comprise at least two dishes, and the at least two dishes may be included in the meal plan in case that one of the at least two dishes is determined in the first set of dishes. The at least one food category may comprise at least one of grains and cereals, fruit, dairy products, nuts, egg, meat, and vegetable.

The user information may comprise at least one of allergens, restricted ingredients, gene/DNA information, a number of meals for a day, height, current weight, expected weight, gender, active level and/or occupational information, location information, diet history data, user pattern data, family history information, and/or stage of the user, wherein the stage of the user may be one of pre-pregnancy, pregnancy, puerperium, or lactation.

The intake category may be one of total calories, macronutrient, micronutrient, and at least one food category.

The meal plan may comprise at least one of a breakfast menu, a lunch menu, a dinner menu, and an extra meal menu, and the extra meal menu may comprise at least one snack for at least one of the breakfast menu, the lunch menu, and the dinner menu.

The at least one snack may comprise at least one snack of dairy products, nuts, and fruit.

The at least one general rule may comprise at least one predetermined rule on energy distribution for a day, weight of each food category for a day, numbers of dishes for at least one of the breakfast menu, the lunch menu, the dinner menu, and the extra meal menu, and/or dish categories for at least one of the breakfast menu, the lunch menu, and the dinner menu, wherein the dish categories may comprise staple dish, soup dish, meat dish, and vegetable dish.

The predetermined rule on the energy distribution may comprise: the actual value of calories of the breakfast menu and the at least one snack for the breakfast menu may be 30 percentage of the target value of the total calories; the actual value of calories of the lunch menu and the at least one snack for the lunch menu may be 40 percentage of the target value of the total calories; and the actual value of calories of the dinner menu and the at least one snack for the dinner menu may be 30 percentage of the target value of the total calories.

The weight of each food category may be obtained based on stage of the user, and the stage of the user may be one of pre-pregnancy, pregnancy, puerperium, or lactation.

The breakfast menu may comprise: the breakfast menu may comprise three dishes.

The lunch menu may comprise: the lunch menu may comprise three dishes.

The dish categories for the lunch menu may comprise a staple dish, one soup dish, a meat dish and/or a vegetable dish.

The dinner menu may comprise: the dinner menu may comprise three dishes.

The dish categories for the dinner menu may comprise a staple dish, a soup dish or a meat dish, and a vegetable dish.

The extra meal menu may comprise two snacks for the breakfast menu, two snacks for the lunch menu, and one snack for the dinner menu.

The dish categories for the extra meal menu may comprise nuts, dairy products, and fruit.

After the determining of the first set of dishes from the predetermined dish set, the method may further comprise: in case that at least one dish in the first set of dishes has a first label, categorising the at least one dish having the first label into an extra meal menu; in case that at least one dish in the first set of dishes has a second label, categorising the at least one dish having the second label into a breakfast menu;

in case that at least one dish in the first set of dishes has a third label, categorising the at least one dish having the third label into a lunch menu; and in case that at least one dish in the first set of dishes has a fourth label, categorising the at least one dish having the fourth label into a dinner menu.

The determining of the second set of dishes from the predetermined dish set may comprise: determining the target value for the each intake category in the extra meal menu based on the at least one general rule, and determining one or more dishes having the first label from the predetermined dish set for the extra meal menu based on the at least one general rule in case that the actual value for the each intake category in the first set of dishes categorised into the extra meal menu is smaller than the target value for the corresponding each intake category in the extra meal menu; determining the target value for the each intake category in the breakfast menu based on the at least one general rule, and determining one or more dishes having the second label from the predetermined dish set for the breakfast menu based on the at least one general rule in case that the actual value for the each intake category in the first set of dishes categorised into the breakfast menu is smaller than the target value for the corresponding each intake category in the breakfast menu; determining the target value for the each intake category in the lunch menu based on the at least one general rule, and determining one or more dishes having the third label from the predetermined dish set for the lunch menu based on the at least one general rule in case that the actual value for the each intake category in the first set of dishes categorised into the lunch menu is smaller than the target value for the corresponding each intake category in the lunch menu; and determining the target value for the each intake category in the dinner menu based on the at least one general rule, and determining one or more dishes having the fourth label from the predetermined dish set for the dinner menu based on the at least one general rule in case that the actual value for the each intake category in the first set of dishes categorised into the dinner menu is smaller than the target value for the corresponding each intake category the dinner menu.

The method may further comprise: in case that the actual intake of one ingredient included in a meal history is greater than or equal to a predefined threshold, sending the user a notification and/or generating the meal plan without a dish containing the one ingredient based on the at least one general rule, wherein the meal history is a daily history or a weekly history.

The diet preference information may be pre-stored or may be obtained from a input module comprising a voice recognition module and/or a text recognition module.

The user information may be pre-stored or may be obtained from a input module comprising a voice recognition module and/or a text recognition module.

The determining of the target value may be further based on standard nutrient recommendations according to the user information of the user.

The determining of the target value may be further based on biometric information, microbiome information, and/or blood glucose information of the user.

The present application provides an apparatus. The apparatus may be configured to comprise at least one processor, wherein the at least one processor may be configured to perform the method mentioned above for generating a meal plan.

The present application provides a storage medium. The storage medium may be configured to store computer instructions, where the instructions may be configured to control at least one processor to perform the method mentioned above for generating a meal plan.

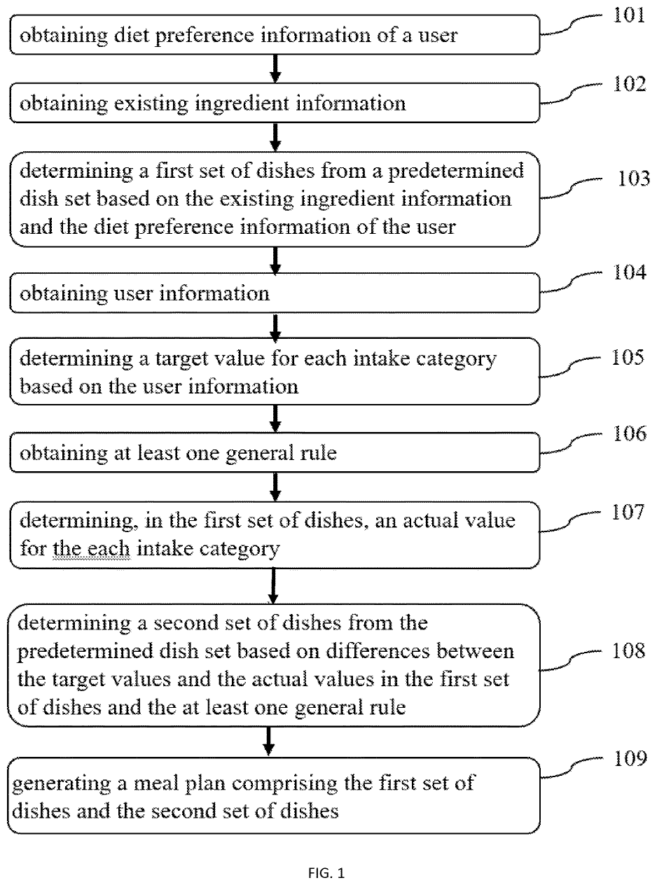

FIG. 1 shows a meal plan generating method. A meal plan may be a plan comprising multiple dishes (e.g., including a first set of dishes and/or a second set of dishes) selected from a predetermined dish set. A dish may a portion of food including drinks, e.g., be solid or mushy food, colloid, liquid/solution, snack and drinks. A dish may be based on one or more food recipe. The predetermined dish set may be obtained from at least one database of different food platforms, such as a cooking app or a food delivery platform, etc. Preferably, the predetermined dish set is obtained from a database of integrating the dishes from the databases of different food platforms (e.g., without overlapping dishes) or of only containing overlapping dishes in the databases of different food platforms.

A meal plan may include at least one of a breakfast menu, a lunch menu, a dinner menu, and an extra meal menu. A breakfast menu may include at least one dish; A lunch menu may include at least one dish; A dinner menu may include at least one dish; and an extra meal menu may include at least one snack. The extra meal menu may comprise at least one snack for at least one of the breakfast menu, the lunch menu, and/or the dinner menu. For example, the extra meal menu may comprise, but is not limited to, nuts, dairy products, and/or fruit. Preferably, the extra meal menu may comprise one snack (with a pre-determined portion in weight or volume) of nuts, one snack (with a pre-determined portion in weight or volume) of dairy products, and three snacks (each with a pre-determined portion in weight or volume) of fruit. The nuts may include, but is not limited to, almond, walnut, peanut, macadamia nut, etc. The dairy products may include, but is not limited to, milk, cheese, yogurt, etc. The fruit may include, but is not limited to, apples, pears, cherry, orange, etc. For example, one snack of nuts may be 100 grams of almond, one snack of dairy products may be 50 grams of blue cheese, and three snacks of fruit may be 80 grams of apple, 80 grams of grapes and 50 grams of orange or 50 ML orange juice. The breakfast menu may comprise, but is not limited to, at least one dish, such as noodles, porridge, buns, dumplings, omelette, cereals, etc. Preferably, the breakfast may comprise three dishes including an egg dish, one noodles/porridge dish, and one cooked wheaten food dish, such as buns, dumplings, etc. The lunch menu and/or the dinner menu may include at least one dish for lunch and/or dinner respectively. For example, the lunch menu and/or the dinner menu may comprise, but is not limited to, at least one of stir-fried dishes, salads, boiled vegetables and meats, steamed dishes, etc. Preferably, the lunch menu and/or the dinner menu may comprise three dishes. Preferably, the lunch menu and/or the dinner menu may include at least one staple dish. For example, the lunch menu and/or the dinner menu may comprise, but is not limited to, at least one of rice, noodles, or cooked wheaten food, etc.

In step 101, the diet preference information of a user is obtained. The diet preference information may include at least one of taste preference, seasoning preference, cooking type preference, ingredients preference, and cuisine preference, etc. Specifically, the taste preference may include, but is not limited to, a preference over the taste of salty, sweet, sour, spicy, bitter and umami. The seasoning preference may include, but is not limited to, a preference over, for example, garlic, ginger, green onion, black pepper, white pepper, mint, etc. The cooking type preference may include, but is not limited to, a method of grilling, frying, boiling, steaming, stirred frying, stewing, etc. The ingredients preference may include a preference over, for example, meat, seafood, egg, bean products, vegetables, etc. The ingredients preference may further include a preference over a specific food category. For example, the user may prefer pork over beef, and/or prefer shrimp over sea-fish, etc. The diet preference information of the user may be obtained via text input or voice input of the user or a selection of the user from a first predetermined database by the user, wherein the first predetermined database may be a predetermined database that stores various information related to diet preference information. The diet preference information may be obtained via inputs from a keyboard, a microphone, a touch screen, or any other input method of an apparatus.

In step 102, the existing ingredient information is obtained. The existing ingredient information may be the information about the ingredient that is available or the ingredient that the user wants to include in the meal plan. The existing ingredient information may be obtained via text input or voice input of the user, or recognized from an image or a selection of the user from a second predetermined database by the user. The second predetermined database may be the same/or different with the first predetermined database.

In step 103, a first set of dishes are determined from a predetermined dish set based on the existing ingredient information and the diet preference information of the user. The predetermined dish set may include pre-determined dishes for the breakfast menu, the lunch menu, the dinner menu, and the extra meal menu. For example, the predetermined dish set may be a set of dishes that are selected from different platforms. Preferably, the predetermined dish set may be a set of dishes that comprises the overlapping dishes in different platforms and/or being scored higher than 8.5. The predetermined dish set may be stored in a third predetermined database, and the third predetermined database may further comprise information on at least one food category and predetermined dish packages. The third predetermined database may be the same/or different with the first predetermined database and/or the second predetermined database. A predetermined dish package may comprise at least two dishes, wherein the at least two dishes may be included in the meal plan in case that one of the at least two dishes has been determined in the first set of dishes. The at least one food category may comprise at least one of grains and cereals, fruit, dairy products, nuts, egg, meat, and vegetable.

After obtaining the diet preference information of the user and the existing ingredient information, the first set of dishes may be determined from the predetermined dish set. Each dish in the first set of dishes may comprise at least one of the one, two or more ingredients comprised in the existing ingredient information. For example, after receiving diet preference information that the user prefers pork over beef, prefers spicy taste, and prefers stirred frying and receiving existing ingredient information that the user have potatoes in the fridge, then dishes that satisfies the conditions (the diet preference information of the user and the existing ingredient information) will be included in the first set of dishes, for example, stirred fried spicy pork with onion and stirred fried sliced potato may be included in the first set of dishes and may be recommended to the user later in the meal plan.

In step 104, user information is obtained. The user information may include information relating to the user, for example, at least one of allergens, restricted ingredients, gene/DNA information, a number of meals for a day, height, current weight, expected weight, gender, active level and/or occupational information, location information, user pattern data, diet history, family history information, and/or stage of the user, for example, the stage being pre-pregnancy, pregnancy, puerperium, or lactation. The user information may further include other information, e.g., the health condition, diseases, body mass index (BMI), age, etc. For example, the diseases may include hyperglycemia, hypoglycemia, hypertension, hypotension, etc.

The user pattern data may comprise data of at least one of user behaviour information with respect to meal plans, e.g., recently selected (or any of saved, searched, commented, viewed longer than a predetermined period, etc.) meal plans or ingredients. The user pattern data may further include other user data when the user uses the present method to obtained meal plan suggestions, e.g., when using an application/software implementing the present method. The location information may be the current location or a previous location of the user, e.g., the birth location, the longest stayed location in the past per-determined period of time, etc. The family history information may be relevant information of the family members of the user, e.g., a family member disease record, the weights of the family members, the diet preference of the family members, etc. The user weight data of the user may comprise the current weight of the user, the weight change data of the user in the past periods, the user weight change data in connection to the meal plans, expected weight, etc. The user weight change data may be very important since it gives hints on dynamic changes of the body/weights such that the meal plans can be suggested/scored more accurately based on the changes. For example, a user who is overweighted and is losing weight too quickly may not be healthy, thus, meals suggested may include more energy/calories temporarily; or a user who is gaining weight very quickly, the energy in the suggested meals should be slowly reduced instead of immediate drop on calories.

The user information may be obtained via text input or voice input of the user or a selection of the user from a fourth predetermined database by the user. For example, the user information may be obtained via at least one of the following ways, e.g., may be obtained via a keyboard, a microphone, a camera, a touch screen, or any other ways. The fourth predetermined database for storing the user information may be the same with the database that stores preference information and/or ingredient information and/or the predetermined dish set or may be different from the database that stores preference information and/or ingredient information and/or the predetermined dish set.

In step 105, a target value (or more target values, in this document, they are used interchangeably) for each intake category is determined based on the user information. The target value may be the standard or recommended intake amount (e.g., mass, volume, or according to any other measurement methods) during a certain period (e.g., a day, a week, a month, the whole pregnancy period, etc). The target value may comprise at least one of a target value for each intake category, and the intake category may be one of calories, macronutrient, micronutrient, and at least one food category, wherein the macronutrient, the micronutrient, and the at least one food category may be predetermined. For example, the food category may comprise at least one of grain/cereal, dairy products, fruit, vegetable, soy products/nuts, sweets/sugar, water, meat, poultry, fish, etc. The macronutrient may comprise at least one of energy, fat, protein, carbohydrate, etc. The micronutrient may comprise at least one of calcium, Fe (iron/ferrum), zinc, folate, vitamin D, vitamin B12, vitamin B6, vitamin C, vitamin B2, Vitamin E, DHA (Docosahexaenoic acid), etc.

The target value may further be determined based on at least one lookup table which provides cross references between user information and standard target values. Data for these lookup tables may be obtained from hospitals, research institutions for nutrition, etc. The target value may further be determined based on at least one trained AI model. Each of the at least one trained AI model may be a model that is able to determine a target value for the user based on the user information. Once the at least one AI model is trained, the at least one trained AI model may output a target value based on the input of the user information. Data for these trained AI models may be obtained from hospitals, research institutions for nutrition, etc.

Preferably, if the user is in a special stage, the target value may further be determined based on the stage information of the user. The stage information may be the information on the stage of the user, for example, the stage being pre-pregnancy, early-pregnancy, pregnancy (e.g., the second trimester and the third trimester), puerperium, or lactation, etc, wherein the puerperium may represent a period during which a female does not feed a baby after parturition. For example, the period may be six months, etc.

For example, the target value of calories may be determined according to at least one of weight gain in the early pregnancy, weight gain in the second and third trimester of pregnancy, and weight gain during lactation in a pre-determined period. The cross-references between target values and the stage information in such an example are shown in Tables 1 to 3, i.e., the target values may be determined based on Tables 1 to 3. The data in Tables 1 to 3 are advantageous and can provide target value suggestions/determinations that are healthy/suitable to the user, which are based on research with a large population sample.

TABLE 1

| | Early | | Early | |
|---|---|---|---|---|
| | pregnancy | | pregnancy | |
| | weight gain | | weight gain | |
| | (kg) (current | | (kg) (current | |
| BMI | weight − | Target | weight − | Target |
| before | pre-pregnancy | value of | pre-pregnancy | value of |
| pregnancy | weight) | calories | weight) | calories |
| BMI ≤ | >2 | 0.9*standard | <0 | 1.1* |
| 18.5 | | target value | | standard |
| | | of calories | | target value |
| | | | | of calories |
| BMI 18.5- | >2 | 0.9*standard | <0 | 1.1* |
| 23.9 | | target value | | standard |
| | | of calories | | target value |
| | | | | of calories |
| BMI 24- | >2 | 0.9*standard | <0 | 1.1* |
| 27.9 | | target value | | standard |
| | | of calories | | target value |
| | | | | of calories |
| BMI ≥ | >2 | 0.9*standard | <0 | 1.1* |
| 28 | | target value | | standard |
| | | of calories | | target value |
| | | | | of calories |

TABLE 2

| | Weight gain | | Weight gain | |
|---|---|---|---|---|
| | in the second | | in the second | |
| | and third | | and third | |
| BMI | trimester of | Target | trimester | Target |
| before | pregnancy | value of | of pregnancy | value of |
| pregnancy | (kg/week) | calories | (kg/week) | calories |
| BMI ≤ 18.5 | >0.56 | 0.9*standard | <0.37 | 1.1* |
| | | target value | | standard |
| | | of calories | | target value |
| | | | | of calories |
| BMI 18.5- | >0.48 | 0.9*standard | <0.26 | 1.1* |
| 23.9 | | target value | | standard |
| | | of calories | | target value |
| | | | | of calories |
| BMI 24-27.9 | >0.37 | 0.9*standard | <0.22 | 1.1* |
| | | target value | | standard |
| | | of calories | | target value |
| | | | | of calories |
| BMI ≥ 28 | >0.3 | 0.9*standard | <0.15 | 1.1* |
| | | target value | | standard |
| | | of calories | | target value |
| | | | | of calories |

TABLE 3

| | Weight gain | | Weight gain | |
|---|---|---|---|---|
| | during | | during | |
| | lactation in | | lactation in | |
| BMI before | 6 months | Target value | 6 months | Target value |
| pregnancy | (kg/month) | of calories | (kg/month) | of calories |
| BMI ≤ 18.5 | >−0.8 | 0.9*standard | >0 | 0.9* |
| | | target value | | standard |
| | | of calories | | target value |
| | | | | of calories |
| BMI 18.5-23.9 | >−0.8 | 0.9*standard | >0 | 0.9* |
| | | target value | | standard |
| | | of calories | | target value |
| | | | | of calories |
| BMI 24-27.9 | >−0.8 | 0.9*standard | >0 | 0.9* |
| | | target value | | standard |
| | | of calories | | target value |
| | | | | of calories |
| BMI ≥ 28 | >−0.8 | 0.9*standard | >0 | 0.9* |
| | | target value | | standard |
| | | of calories | | target value |
| | | | | of calories |

Preferably, the target value may further be determined based on the active level information and/or occupational information of the user. The active level information may be the information indicating the user's physical active level. The active level may be classified into three different active levels (i.e., low-active, moderate-active and high-active) and may have different influences on the target values. The low-active level may be determined for users whose calory consumption is below or equal to a first calory threshold; the moderate-active level may be determined for users whose calory consumption is higher than the first calory threshold and below or equal to a second calory threshold; the high-active level may be determined for users whose calory consumption is higher than the second calory threshold. More levels may be defined in a similar way when determining the target value. The occupational information may be used to reflect the active level information, and thus may be used to determine a target value. For example, if a user is a man and he is an athlete, the target value of the user may be much higher than the standard recommendation for the actual values. An example of a cross reference between target values and the active levels for males is shown in Table 4 according to research in a large population sample, based on which the target values may be determined.

TABLE 4

| Age from 18-49 | Low active | Moderate active | High active |
|---|---|---|---|
| (Male) | level | level | level |
| Energy | 2250 | 2600 | 3000 |
| (kcal/day) | | | |
| Fat (g/day) | 62.5 | 72.22222222 | 83.3333333 |
| Protein (g/day) | 84.375 | 97.5 | 112.5 |
| Carbohydrates | 337.5 | 390 | 450 |
| (g/day) | | | |

In Table 4, the low active level may represent that a user's occupation does not involve many physical activities. For example, the low active level may represent the active level of a user who works in an office and always sits for a long period, and/or the active level of a user who only does limited sports like yoga or walking occasionally. The moderate active level may represent that a user's occupation needs standing or walking. For example, the moderate active level may represent the active level of a user who may sometimes jug, dance, do housework, etc. The high active level may represent that a user's occupation involves many physical activities. For example, the high active level may represent a user who may be a heavy worker and/or always participating in sports such as cycling, sprinting, skiing, etc.

As an example, for females, the cross-references among target values, stage information, and the active level information are shown in Tables 5-9 according to research in a large population sample, which the target values can be determined based on

TABLE 5

| | Low active | Moderate active | High active |
|---|---|---|---|
| Pre-pregnancy | level | level | level |
| Energy (kcal/day) | 1800 | 2100 | 2400 |
| Fat (g/day) | 50 | 58.33333333 | 66.66666667 |
| Protein (g/day) | 67.5 | 78.75 | 90 |
| Carbohydrates | 270 | 315 | 360 |
| (g/day) | | | |

TABLE 6

| | Low active | Moderate active | High active |
|---|---|---|---|
| Early-pregnancy | level | level | level |
| Energy (kcal/day) | 1800 | 2100 | 2400 |
| Fat (g/day) | 50 | 58.33333333 | 66.66666667 |
| Protein (g/day) | 67.5 | 78.75 | 90 |
| Carbohydrates | 270 | 315 | 360 |
| (g/day) | | | |

TABLE 7

| the second | | | |
|---|---|---|---|
| trimester of | Low active | Moderate active | High active |
| pregnancy | level | level | level |
| Energy (kcal/day) | 2100 | 2400 | 2700 |
| Fat (g/day) | 58.33333333 | 66.66666667 | 75 |
| Protein (g/day) | 78.75 | 90 | 101.25 |
| Carbohydrates | 315 | 360 | 405 |
| (g/day) | | | |

TABLE 8

| the third trimester | Low active | Moderate active | High active |
|---|---|---|---|
| of pregnancy | level | level | level |
| Energy (kcal/day) | 2250 | 2550 | 2850 |
| Fat (g/day) | 62.5 | 70.83333333 | 79.16666667 |
| Protein (g/day) | 84.375 | 95.625 | 106.875 |
| Carbohydrates | 337.5 | 382.5 | 427.5 |
| (g/day) | | | |

TABLE 9

| | Low active | Moderate active | High active |
|---|---|---|---|
| lactation | level | level | level |
| Energy (kcal/day) | 2300 | 2600 | 2900 |
| Fat (g/day) | 63.88888889 | 72.22222222 | 80.55555556 |
| Protein (g/day) | 86.25 | 97.5 | 108.75 |
| Carbohydrates | 345 | 390 | 435 |
| (g/day) | | | |

As an example, for females, the cross-references among target values, stage information, and the active level information are shown in Tables 10-16 according to research in a large population sample, which the target values can be determined based on. In the table, ht(m)² represents the height of the user (in meters) squared.

TABLE 10

| | Target value of | Target value of | Target value of |
|---|---|---|---|
| Pre- | calories for low | calories for moderate | calories for high |
| pregnancy | active level | active level | active level |
| BMI ≤ 18.5 | 21.2*ht(m)²*37.5 | 21.2*ht(m)²*37.5/1.5*1.75 | 21.2*ht(m)²*37.5/1.5*2 |
| BMI 18.5-23.9 | 21.2*ht(m)²*32.5 | 21.2*ht(m)²*32.5/1.5*1.75 | 21.2*ht(m)²*32.5/1.5*2 |
| BMI 24-27.9 | 21.2*ht(m)²*27.5 | 21.2*ht(m)²*27.5/1.5*1.75 | 21.2*ht(m)²*27.5/1.5*2 |
| BMI ≥ 28 | 21.2*ht(m)²*27.5 | 21.2*ht(m)²*27.5/1.5*1.75 | 21.2*ht(m)²*27.5/1.5*2 |

TABLE 11

| | Target value of | Target value of | Target value of |
|---|---|---|---|
| Early- | calories for low | calories for moderate | calories for high |
| pregnancy | active level | active level | active level |
| BMI ≤ 18.5 | 21.2*ht(m)²*37.5 | 21.2*ht(m)²*37.5/1.5*1.75 | 21.2*ht(m)²*37.5/1.5*2 |
| BMI 18.5-23.9 | 21.2*ht(m)²*32.5 | 21.2*ht(m)²*32.5/1.5*1.75 | 21.2*ht(m)²*32.5/1.5*2 |
| BMI 24-27.9 | 21.2*ht(m)²*27.5 | 21.2*ht(m)²*27.5/1.5*1.75 | 21.2*ht(m)²*27.5/1.5*2 |
| BMI ≥ 28 | 21.2*ht(m)²*27.5 | 21.2*ht(m)²*27.5/1.5*1.75 | 21.2*ht(m)²*27.5/1.5*2 |

TABLE 12

| the second | Target value of | Target value of | Target value of |
|---|---|---|---|
| trimester | calories for low | calories for moderate | calories for high |
| of pregnancy | active level | active level | active level |
| BMI ≤ 18.5 | 21.2*ht(m)²*37.5 + | 21.2*ht(m)²*37.5/1.5*1.75 + | 21.2*ht(m)²*37.5/1.5*2 + |
| | 300 | 300 | 300 |
| BMI 18.5-23.9 | 21.2*ht(m)²*32.5 + | 21.2*ht(m)²*32.5/1.5*1.75 + | 21.2*ht(m)²*32.5/1.5*2 + |
| | 300 | 300 | 300 |
| BMI 24-27.9 | 21.2*ht(m)²*27.5 + | 21.2*ht(m)²*27.5/1.5*1.75 + | 21.2*ht(m)²*27.5/1.5*2 + |
| | 300 | 300 | 300 |
| BMI ≥ 28 | 21.2*ht(m)²*27.5 + | 21.2*ht(m)²*27.5/1.5*1.75 + | 21.2*ht(m)²*27.5/1.5*2 + |
| | 300 | 300 | 300 |

TABLE 13

| the third | Target value of | Target value of | Target value of |
|---|---|---|---|
| trimester | calories for low | calories for moderate | calories for high |
| of pregnancy | active level | active level | active level |
| BMI ≤ 18.5 | 21.2*ht(m)²*37.5 + | 21.2*ht(m)²*37.5/1.5*1.75 + | 21.2*ht(m)²*37.5/1.5*2 + |
| | 450 | 450 | 450 |
| BMI 18.5-23.9 | 21.2*ht(m)²*32.5 + | 21.2*ht(m)²*32.5/1.5*1.75 + | 21.2*ht(m)²*32.5/1.5*2 + |
| | 450 | 450 | 450 |
| BMI 24-27.9 | 21.2*ht(m)²*27.5 + | 21.2*ht(m)²*27.5/1.5*1.75 + | 21.2*ht(m)²*27.5/1.5*2 + |
| | 450 | 450 | 450 |
| BMI ≥ 28 | 21.2*ht(m)²*27.5 + | 21.2*ht(m)²*27.5/1.5*1.75 + | 21.2*ht(m)²*27.5/1.5*2 + |
| | 450 | 450 | 450 |

TABLE 14

| lactation | Target value of | Target value of | Target value of |
|---|---|---|---|
| within 6 | calories for low | calories for moderate | calories for high |
| months | active level | active level | active level |
| BMI ≤ 18.5 | 21.2*ht(m)²*37.5 + | 21.2*ht(m)²*37.5/1.5*1.75 + | 21.2*ht(m)²*37.5/1.5*2 + |
| | 500 | 500 | 500 |
| BMI 18.5-23.9 | 21.2*ht(m)²*32.5 + | 21.2*ht(m)²*32.5/1.5*1.75 + | 21.2*ht(m)²*32.5/1.5*2 + |
| | 500 | 500 | 500 |
| BMI 24-27.9 | 21.2*ht(m)²*27.5 + | 21.2*ht(m)²*27.5/1.5*1.75 + | 21.2*ht(m)²*27.5/1.5*2 + |
| | 300 | 300 | 300 |
| BMI ≥ 28 | 21.2*ht(m)²*27.5 + | 21.2*ht(m)²*27.5/1.5*1.75 + | 21.2*ht(m)²*27.5/1.5*2 + |
| | 500 | 500 | 500 |

TABLE 15

| | Target value of | Target value of | Target value of |
|---|---|---|---|
| | calories for low | calories for moderate | calories for high |
| Puerperium | active level | active level | active level |
| BMI ≤ 18.5 | 21.2*ht(m)²*37.5 + | 21.2*ht(m)²*37.5/1.5*1.75 + | 21.2*ht(m)²*37.5/1.5*2 + |
| | 500 | 500 | 500 |
| BMI 18.5-23.9 | 21.2*ht(m)²*32.5 + | 21.2*ht(m)²*32.5/1.5*1.75 + | 21.2*ht(m)²*32.5/1.5*2 + |
| | 500 | 500 | 500 |
| BMI 24-27.9 | 21.2*ht(m)²*27.5 + | 21.2*ht(m)²*27.5/1.5*1.75 + | 21.2*ht(m)²*27.5/1.5*2 + |
| | 300 | 300 | 300 |
| BMI ≥ 28 | 21.2*ht(m)²*27.5 + | 21.2*ht(m)²*27.5/1.5*1.75 + | 21.2*ht(m)²*27.5/1.5*2 + |
| | 500 | 500 | 500 |

TABLE 16

| lactation after | | | |
|---|---|---|---|
| 6 months or | Target value of | Target value of | Target value of |
| mixed feeding | calories for low | calories for moderate | calories for high |
| period | active level | active level | active level |
| BMI ≤ 18.5 | 21.2*ht(m)²*37.5 + | 21.2*ht(m)²*37.5/1.5*1.75 + | 21.2*ht(m)²*37.5/1.5*2 + |
| | lactation | lactation | lactation |
| | amount(ml)*0.85-170 | amount(ml)*0.85-170 | amount(ml)*0.85-170 |
| BMI 18.5-23.9 | 21.2*ht(m)²*32.5 + | 21.2*ht(m)²*32.5/1.5*1.75 + | 21.2*ht(m)²*32.5/1.5*2 + |
| | lactation | lactation | lactation |
| | amount(ml)*0.85-170 | amount(ml)*0.85-170 | amount(ml)*0.85-170 |
| BMI 24-27.9 | 21.2*ht(m)²*27.5 + | 21.2*ht(m)²*27.5/1.5*1.75 + | 21.2*ht(m)²*27.5/1.5*2 + |
| | lactation | lactation | lactation |
| | amount(ml)*0.85-170 | amount(ml)*0.85-170 | amount(ml)*0.85-170 |
| BMI ≥ 28 | 21.2*ht(m)²*27.5 + | 21.2*ht(m)²*27.5/1.5*1.75 + | 21.2*ht(m)²*27.5/1.5*2 + |
| | lactation | lactation | lactation |
| | amount(ml)*0.85-170 | amount(ml)*0.85-170 | amount(ml)*0.85-170 |

When determining one or more target values of the user, target values for different intake categories may be determined in a separate AI model, or target values for different intake categories may be determined in the same AI model, or some of the target values for different intake categories may be determined in different AI models and some of the target values for different intake categories may be determined in one same AI model.

In step 106, at least one general rule is obtained. The at least one general rule may comprise at least one predetermined rule on at least one of energy distribution for a day, weight of each food category for a day, number of dishes for at least one of the breakfast menu, the lunch menu, the dinner menu, and the extra meal menu, and dish categories for at least one of the breakfast menu, the lunch menu, and the dinner menu, and wherein the dish categories may comprise staple dish, soup dish, meat dish, and vegetable dish. A meat dish may comprise meat only or a mixture of meat and vegetables. A vegetable dish may only comprise vegetables.

For example, the energy distribution for a day may be: the actual value of calories of the breakfast menu and the at least one snack for the breakfast menu maybe 30 percent of the target value of the total calories; the actual value of calories of the lunch menu and the at least one snack for the lunch menu maybe 40 percent of the target value of the total calories; and the actual value of calories of the dinner menu and the at least one snack for the dinner menu maybe 30 percent of the target value of the total calories, wherein the total calories may be a sum of calories of a day, including the calories of all the dishes in the extra meal menu, the breakfast menu, the lunch menu, and the dinner menu.

For example, a meal plan may include at least one of a breakfast menu, a lunch menu, a dinner menu, and an extra meal menu, wherein the extra meal menu may comprise at least one snack for at least one of the breakfast menu, the lunch menu, and the dinner menu. The breakfast menu may comprise, but is not limited to, at least one dish, such as noodles, porridge, buns, dumplings, omelette, cereals, etc. Preferably, the breakfast may comprise three dishes including an egg dish, a noodles/porridge dish, and a cooked wheaten food dish, such as buns, dumplings, etc. The lunch menu and/or the dinner menu may include at least one dish for lunch and/or dinner. For example, the lunch menu and/or the dinner menu may comprise, but is not limited to, at least one of stir-fried dishes, salads, boiled vegetables and meats, steamed dishes, etc. Preferably, the lunch menu and/or the dinner menu may comprise three dishes. Preferably, the lunch menu and/or the dinner menu may include at least one staple dish. For example, the lunch menu may comprise, but is not limited to, one staple dish, one soup dish, one meat dish or one vegetable dish. For example, the dinner menu may comprise, but is not limited to, one staple dish, one soup dish or one meat dish, and one vegetable dish. A meat dish may comprise meat only or a mixture of meat and vegetables. A vegetable dish may only comprise vegetables. The extra meal menu may include two snacks for the breakfast menu, two snacks for the lunch menu, and one snack for the dinner menu. Preferably, the extra meal menu may comprise one snack of nuts, one snack of dairy products, and three snacks of fruit.

For example, the weight of each food category for a day may be obtained based on the user information, preferably may be further based on the stage of the user and/or occupational information. The stage of the user may be, for example, the stage being pre-pregnancy, early-pregnancy, pregnancy (e.g., the second trimester and the third trimester), puerperium, or lactation, etc, wherein the puerperium may represent a period during which a female does not feed a baby after parturition, for example, 6 months. As an example, the weight of each food category for a day may be determined as shown in Table 17 based on research with a large population sample.

TABLE 17

| | | | the second | the third | |
|---|---|---|---|---|---|
| | | Early- | trimester of | trimester of | |
| | | pregnancy | pregnancy | pregnancy | Lactation |
| | Adult (g) | (g) | (g) | (g) | (g) |
| Grain | 250-300 | 250-300 | 275-325 | 300-350 | 300-350 |
| Meat | 130-180 | 130-180 | 150-200 | 200-250 | 200-250 |
| Fish | 40-65 | 40-65 | 50-75 | 75-100 | 75-100 |
| Egg | 40-60 | 40-60 | 40-60 | 40-60 | 40-60 |
| Dairy products | 300 | 300 | 300-500 | 300-500 | 300-500 |
| Fruit | 250-350 | 250-350 | 200-400 | 200-400 | 200-400 |
| vegetable | 300-500 | 300-500 | 300-500 | 300-500 | 400-500 |
| Beans and nuts | 20-30 | 20-30 | 24-36 | 24-36 | 28-42 |
| Water | 1360- | 1500-1700 | 1700-1900 | 1700-1900 | 2100-2300 |
| | 2040 (for | | | | |
| | male); | | | | |
| | 1500- | | | | |
| | 1700 (for | | | | |
| | female) | | | | |

In step 107, an actual value for the each intake category in the first set of dishes is determined. The actual value for the each intake category in the first set of dishes may be determined based on the portion in weight or volume of each intake category that is contained in the first set of dishes.

In step 108, a second set of dishes is determined from the predetermined dish set based on differences between the target values and the actual values in the first set of dishes and the at least one general rule. The second set of dishes may be a set of dishes that are determined from the predetermined dish set to compensate for the intake differences between the target values and the actual values in the first set of dishes based on the at least one general rule. The predetermined dish set may be stored in the third predetermined database which includes, for example, a set of dishes for a breakfast menu, a set of dishes for a lunch menu, a set of dishes for a dinner menu, and a set of dishes/snacks for an extra meal menu, from different platforms. The predetermined dish set for determining the second set of dishes may be the same as the predetermined dish set for determining the first set of dishes.

In step 109, a meal plan comprising the first set of dishes and the second set of dishes is generated.

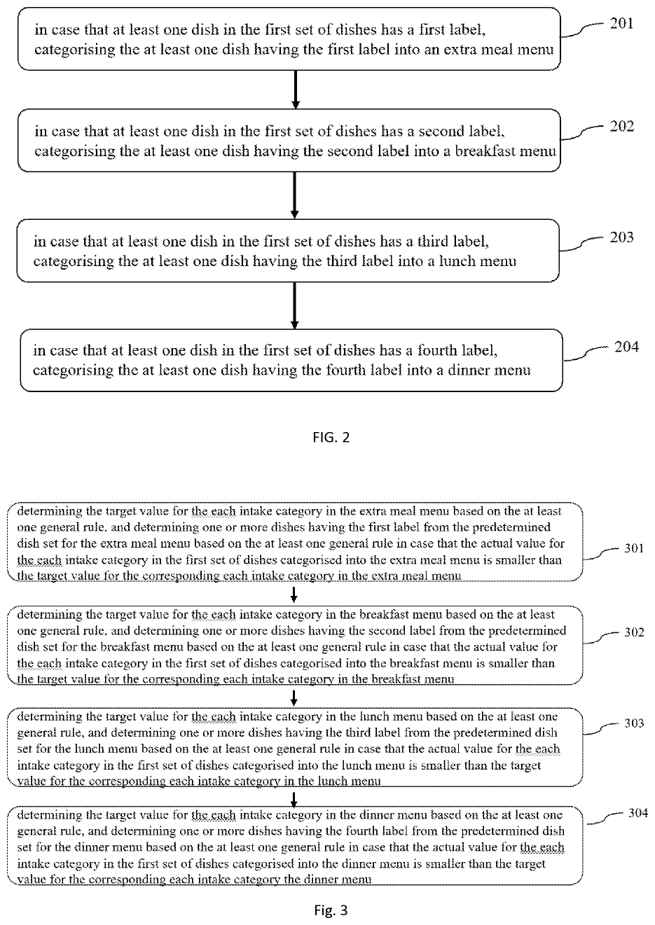

FIG. 2 shows a method of categorising the first set of dishes after the determining of the first set of dishes from the predetermined dish set (e.g., step 103), which may be used when determining whether the rule(s) is met. The third predetermined database storing the first set of dishes may further include label information. The label information may be the information of at least one label. Each dish in the predetermined dish set has a corresponding label and the label may be used to indicate whether a dish can be categorised into the breakfast menu, the lunch menu, the dinner menu, or the extra meal menu. For example, the first label may be used to indicate that a corresponding dish may be categorised into the extra meal menu, the second label may be used to indicate that a corresponding dish may be categorised into the breakfast menu, the third label may be used to indicate that a corresponding dish may be categorised into the lunch menu, and the fourth label may be used to indicate that a corresponding dish may be categorised into the dinner menu.

In step 201, in case that at least one dish in the first set of dishes has a first label, the at least one dish having the first label may be categorised into an extra meal menu.

In step 202, in case that at least one dish in the first set of dishes has a second label, the at least one dish having the second label may be categorised into a breakfast menu.

In step 203, in case that at least one dish in the first set of dishes has a third label, the at least one dish having the third label may be categorised into a lunch menu.

In step 204, in case that at least one dish in the first set of dishes has a fourth label, the at least one dish having the fourth label may be categorised into a dinner menu.

FIG. 3 shows a method of determining a second set of dishes based on the first set of dishes and at least one general rule (as an example of step 108, which is combinable with any other steps in FIG. 1), where the first set of dishes may be determined based on the existing ingredient information and the user preference information (e.g., step 103). Specifically, after receiving the existing ingredient information and the user preference information, a first set of dishes may be determined from the third predetermined database, and the third predetermined database may store a predetermined dish set. For example, the predetermined dish set may include a set of dishes for a breakfast menu, a set of dishes for a lunch menu, a set of dishes for a dinner menu, and a set of dishes for an extra meal menu. However, the actual value of at least one intake category in the first set of dishes may not satisfy the target value for the at least one intake category. In that case, a second set of dishes may be determined as follows in order to meet the target value for each intake category (e.g., as in step 108).

In step 301, in case that the actual value for at least one intake category in the first set of dishes categorised into the extra meal menu is smaller than the target value for the corresponding at least one intake category in the extra meal menu by a first threshold, a second set of dishes may be determined to include at least one snack based on an intake difference between the actual value and the corresponding target value for the at least one intake category and the at least one general rule as defined in step 106. The first threshold may be predetermined or may be input by the user.

For example, if the first set of dishes only includes one snack of fruit, then one snack of dairy products, one snack of nuts, and two more snacks of fruit may be determined for the second set of dishes for the extra meal menu. The intake difference between the actual values of the dishes in the extra meal menu and the corresponding target value may below or equal to the first threshold.

In step 302, in case that the actual value for at least one intake category in the first set of dishes categorised into the breakfast menu is smaller than the target value for the corresponding at least one intake category in the breakfast menu, one or more dishes having the first label may further be determined from the predetermined dish set for the breakfast menu based on an intake difference between the at least one target value and the corresponding actual values for the at least one intake category and the at least one general rule as defined in step 106.

For example, if the first set of dishes contains one or more dishes, for example, buns, porridge, cereals, etc., at least one dish having the second label from the first set of dishes may be categorised into the breakfast menu. In case that the actual value of calories of the at least one dish having the second label from the first set of dishes is smaller than the corresponding target value of calories by a second threshold, a second set of dishes having the second label may be determined to further include one or more dishes for breakfast based on an intake difference between the actual value and the corresponding target value of calories and the at least one general rule as defined in step 106. The second threshold may be predetermined or may be input by the user.

For example, if the first set of dishes includes meat buns but doesn't include any other dishes for breakfast, then two more dishes may be determined for the second set of dishes for breakfast according to the at least one general rule as defined in step 106, such that the intake difference between the actual values of the dishes in the breakfast menu and the corresponding target value may below or equal to the second threshold.

In step 303, in case that the actual value for at least one intake category in the first set of dishes categorised into the lunch menu is smaller than the target value for the corresponding at least one intake category in the lunch menu, one or more dishes having the third label may further be determined from the predetermined dish set for the lunch menu based on an intake difference between the at least one target value and the corresponding actual values for the at least one intake category and the at least one general rule general as defined in step 106.

For example, if the first set of dishes contains one or more dishes, for example, Mapo tofu and Peking duck, at least one dish having the third label from the first set of dishes may be categorised into the lunch menu. In case that the actual values of calories of the at least one dish having the third label from the first set of dishes is smaller than the corresponding target value of calories by a third threshold, a second set of dishes may be determined to further include one or more dishes having the third label for the lunch menu based on an intake difference between the actual value and the corresponding target value of calories and the at least one general rule as defined in step 106. The third threshold may be predetermined or may be input by the user.

For example, if the first set of dishes includes Peking duck, Mapo tofu, and stirred fired cabbage but doesn't include any other dishes having the third label, then one soup and one staple dish having the third label may be determined for the second set of dishes, such that the intake difference between the actual values of the dishes in the lunch menu and the corresponding target value may below or equal to the third threshold, wherein the lunch menu comprises one soup dish and one staple dish from the second set of dishes together with the one dish, for example, one meat dish or one vegetable dish, chosen from Peking duck, Mapo tofu, and stirred fired cabbage from the first set of dishes. A meat dish may comprise meat only or a mixture of meat and vegetables. A vegetable dish may only comprise vegetables.

In step 304, in case that the actual value for at least one intake category in the first set of dishes categorised into the dinner menu is smaller than the target value of calories of the dinner menu, one or more dishes having the fourth label may further be determined from the predetermined dish set for the dinner menu based on an intake difference between the at least one target value and the corresponding actual values for the at least one intake category and the at least one general rule as defined in step 106.

For example, if the first set of dishes contain one or more dishes, for example, Mapo tofu, Peking duck, stirred fired cabbage, etc., at least one dish having the fourth label from the first set of dishes may be categorised into the dinner menu. The fourth threshold may be predetermined or may be input by the user.

For example, if the first set of dishes includes Peking duck, Mapo tofu, and stirred fired cabbage but doesn't include any other dishes for dinner, since two of the Peking duck, Mapo tofu, and stirred fired cabbage, for example, Peking duck and Mapo tofu, have been chosen as dishes for lunch, the remaining one dish, i.e., stirred fired cabbage, may be chosen for dinner. To meet the at least one general rule, for example, dinner including one meat dish or one vegetable dish, and one soup dish and one staple dish, then one staple dish and one soup dish having the fourth label may be further determined to be included in the second set of dishes for dinner, such that the intake difference between the actual values of the dishes in the dinner menu and the corresponding target value may below or equal to the fourth threshold.

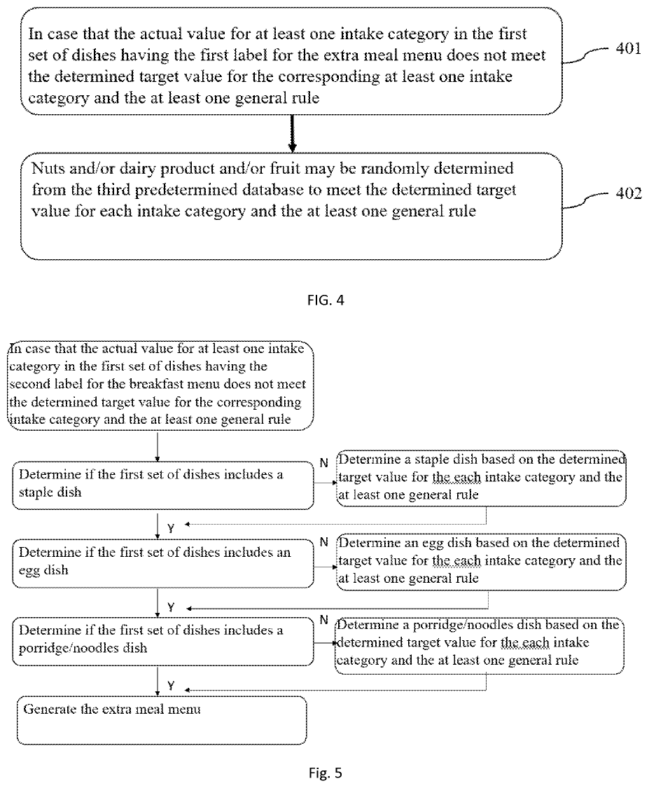

FIG. 4 shows a detailed method of determining the extra meal menu (as an example of step 301). The extra meal menu may comprise at least one snack for a breakfast, lunch, and/or dinner. In case that the actual value for at least one intake category in the first set of dishes having the first label does not meet the determined target value for the corresponding at least one intake category and the at least one general rule as defined in step 106, nuts, and/or dairy products, and/or fruit may be randomly determined from the third predetermined database to meet the determined target value for the each intake category and the at least one general rule (as shown in 401 and 402).

For example, if the ingredient information includes nuts but not dairy products and fruit, the first set of dishes may not include dairy products and fruit. To meet the at least one general rule as defined in step 106 and the target value for each intake category, one snack of dairy products and three snacks of fruit may be randomly determined from the third predetermined database. Then the recommended extra meal menu may comprise the one snack of nuts, the one snack of dairy products, and the three snacks of fruit. The actual value for the each intake category in the extra meal menu may meet the determined target value for the each intake category and the at least one general rule as defined in step 106.

FIG. 5 shows a detailed method of determining the breakfast menu (as an example of step 302).

In case that the actual value for at least one intake category in the first set of dishes for the breakfast menu does not meet the determined target value for the each intake category and the at least one general rule as defined in step 106, one or more dishes having the second label for the breakfast menu may be further determined from the third predetermined database to be included in the second set of dishes, so that to meet the determined target value for the each intake category and the at least one general rule. For example, if the first set of dishes includes one meat bun dish but does not include any egg dish and any porridge/noodles dish, then one egg dish and one porridge/noodles dish may be included in the second set of dishes to meet the determined target value for the each intake category and the at least one general rule. Then the breakfast menu may comprise one meat bun dish, one egg dish, and one porridge/noodles dish. The at least one actual value of the breakfast menu may meet the determined target value for the each intake category and the at least one general rule.

When determining dishes for the breakfast menu, whether one staple dish is included in the first set of dishes may be first determined. The staple dish having a second label for breakfast may be a dish, such as meat buns, vegetable buns, bread, etc. If no staple dish having the second label for breakfast has been included in the first set of dishes, one staple dish having the second label for the breakfast menu may be randomly determined from the third predetermined database to be included in the second set of dishes, so that to meet the determined target value for the each intake category and the at least one general rule as defined in step 106. If one staple dish having the second label for breakfast is included in the first set of dishes, the actual value of calories for the one staple dish may be calculated. If the actual value of calories for the one staple dish having the second label is larger than the target value of calories for the breakfast menu, the amount (e.g., weight) of the one staple dish may be adjusted until the actual value of calories for the one staple dish is smaller than the determined target value of calories for the breakfast menu. If the actual value of calories for the one staple dish is smaller than the determined target value of calories for the breakfast menu, whether one egg dish having the second label has been included in the first set of dishes may be determined as follows.

If no egg dish having the second label for the breakfast menu has been included in the first set of dishes, one egg dish having the second label may be randomly determined for the breakfast menu from the second predetermined database to be included in the second set of dishes, so that to meet the determined target value of calories for the breakfast menu and the at least one general rule as defined in step 106. If one egg dish having the second label for breakfast has been included in the first set of dishes, the actual value of calories for the one staple dish and the one egg dish having the second label may be calculated. If the actual value of calories for the one staple dish and one egg dish having the second label is larger than the determined target value of calories for the breakfast menu, the amount (e.g., weight) of the one staple dish and the one egg dish having the second label may be adjusted until the actual value of calories for the one staple dish and the one egg dish having the second label is smaller than the determined target value of calories for the breakfast menu. If the actual value of calories for the one staple dish and the one egg dish having the second label is smaller than the determined target value of calories for the breakfast menu, whether one porridge/noodles dish having the second label has been included in the first set of dishes may be determined as follows.

If no porridge/noodles dish having the second label for breakfast has been included in the first set of dishes, one porridge/noodles dish having the second label for the breakfast menu may be randomly determined from the third predetermined database to be included in the second set of dishes, so that to meet the determined target value of calories for the breakfast menu and the at least one general rule as defined in step 106. If one porridge/noodles dish having the second label for breakfast has been included in the first set of dishes, the amount (e.g., weight) of the one porridge/noodles dish having the second label is determined based on the actual value of calories for the one staple dish and the one egg dish and the determined target value for each intake category for the breakfast menu, for example, the amount of the one porridge/noodles dish is determined based on the intake difference between the actual value of calories for the one staple dish and the one egg dish having the second label and the determined target value of calories for the breakfast menu and the at least one general rule.

After determining the dishes including one staple dish, one egg dish, and one porridge/noodles dish having the second label for the breakfast menu, the determined dishes may be added to the meal plan as the breakfast menu and may be recommended to the user.

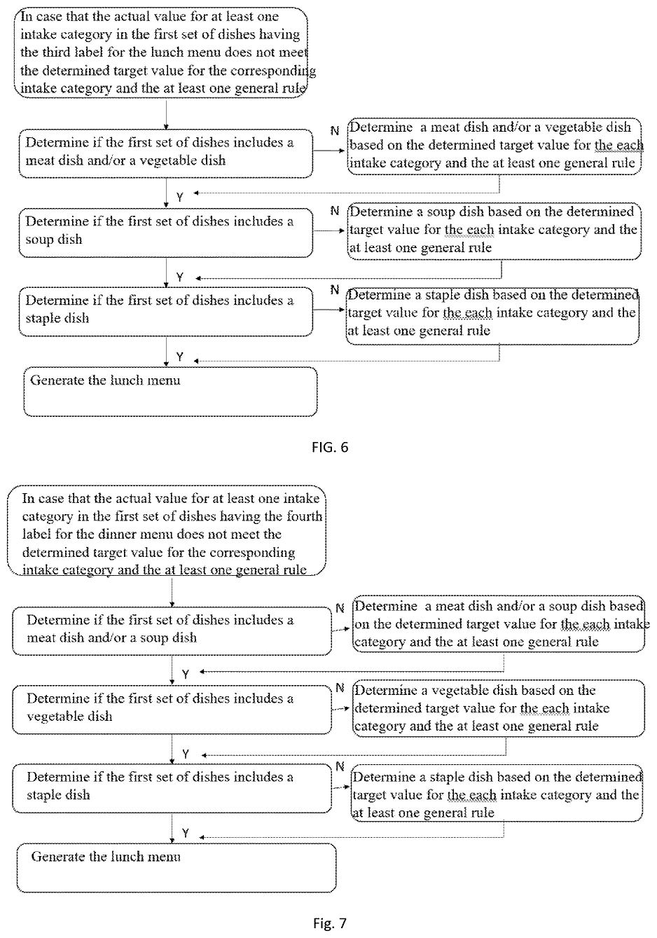

FIG. 6 shows a detailed method of determining the lunch menu (as an example of step 303). In case that the actual value for at least one intake category in the first set of dishes for the lunch menu does not meet the determined target value for the at least one intake category and the at least one general rule as defined in step 106, one or more dishes having a third label for the lunch menu may further be randomly determined from the third predetermined database to be included in the second set of dishes, so that to meet the determined target value for the each intake category and the at least one general rule as defined in step 106. For example, if the first set of dishes includes two meat dishes but does not include any vegetable dish or soup dish, then one staple dish and one soup dish having the third label may further be randomly determined to be included in the second set of dishes, so that to meet the determined target value for the each intake category and the at least one general rule. Then the lunch menu may comprise the one meat dish, the one staple dish, and the one soup dish. The actual value for the each intake category for the lunch menu may meet the determined target value for the each intake category and the at least one general rule as defined in step 106. Preferable, the lunch menu may comprise one staple dish, one soup dish, and one meat dish or a vegetable dish having the third label.

When determining dishes for the lunch menu, whether one meat dish or one vegetable dish having the third label has been included in the first set of dishes may be first determined. A meat dish may comprise meat only or a mixture of meat and vegetables. A vegetable dish may only comprise vegetables. The meat dish having the third label may be, for example, Peking duck, etc. The vegetable dish having the third label may be Mapo tofu, stirred fired cabbage, etc. If no meat dish or vegetable dish having the third label for the lunch menu has been included in the first set of dishes, one meat dish or one vegetable dish having the third label may be further randomly determined for the lunch menu from the third predetermined database to be included in the second set of dishes, so that to meet the determined target value for the each intake category for the lunch menu and the at least one general rule as defined in step 106. If the actual value of calories for the one meat dish or the vegetable dish having the third label is larger than the target value of calories for the lunch menu, the amount (e.g., weight) of the one meat dish or vegetable dish having the third label may be adjusted until the actual value of calories for the one meat dish or one vegetable dish having the third label is smaller than the determined target value of calories for the lunch menu. If one meat dish or one vegetable dish having the third label for the lunch menu has been included in the first set of dishes, the actual value of calories for the one meat dish or one vegetable dish having the third label may be calculated. If the actual value of calories for the one meat dish or one vegetable dish having the third label is smaller than the determined target value of calories for the lunch menu, whether one soup dish having the third label has been included in the first set of dishes may be determined as follows.

If no soup dish having the third label for the lunch menu has been included in the first set of dishes, one soup dish having the third label may be further randomly determined for the lunch menu from the third predetermined database to be included in the second set of dishes, so that to meet the determined target value for each intake category for the lunch menu and the at least one general rule as defined in step 106. If one soup dish having the third label for the lunch menu has been included in the first set of dishes, the actual value of calories for the one meat dish or one dish and the one soup dish having the third label may be calculated. If the actual value of calories for the one meat dish or one vegetable dish and the one soup dish having the fourth label is larger than the determined target value of calories in the lunch menu, the amount (e.g., weight) of the one meat dish or one vegetable dish and the soup dish having the third label may be adjusted until the actual value of calories for the one meat dish or one vegetable dish and the one soup dish having the third label is smaller than the determined target value of calories for the lunch menu. If the actual value of calories for the one meat dish or one vegetable dish and the one soup dish having the third label is smaller than the determined target value of calories for the lunch menu, whether one staple dish having the third label has been included in the first set of dishes may be determined as follows.

If no staple dish having the third label for the lunch menu has been included in the first set of dishes, one staple dish having the third label may further be randomly determined for the lunch menu from the third predetermined database to be included in the second set of dishes, so that to meet the determined target value for each intake category for the lunch menu and the at least one general rule as defined in step 106. If one staple dish having the third label for the lunch menu has been included in the first set of dishes, the amount of the one staple dish having the third label is determined based on the actual values of calories of the one meat dish or the one vegetable dish and the one soup dish and the determined target value for each intake category for the lunch menu, for example, the amount (e.g., weight) of the one staple dish having the third label is determined based on the intake difference between the sum of the actual values of calories of the one meat dish or the one vegetable dish and the one soup dish that have the third label and the determined target value for each intake category for the lunch menu and the at least one general rule as defined in step 106.

After determining the dishes including one staple dish, one meat dish or one vegetable dish, and one soup dish having the third label for the lunch menu, the dishes may be added to the meal plan as the lunch menu and may be recommended to the user.

FIG. 7 shows a detailed method of determining the dinner menu (as an example of step 304). In case that the actual value for at least one intake category in the first set of dishes for the dinner menu does not meet the determined target value for the each intake category and the at least one general rule as defined in step 106, one or more dishes having a fourth label may be further randomly determined for the dinner menu from the third predetermined database to be included in the second set of dishes, so that to meet the determined target value for the each intake category and the at least one general rule as defined in step 106. For example, if the first set of dishes includes two meat dishes but does not include any vegetable dish or soup dish, then one vegetable dish and one staple dish having the fourth label may further be randomly determined to be included in the second set of dishes, so that to meet the determined target value for the each intake category and the at least one general rule. Then the dinner menu may comprise the one meat dish or one soup dish, the one vegetable dish, and the one staple dish that have the fourth label. The actual value for the each intake category in the dinner menu may meet the determined target value for the each intake category and the at least one general rule.

When determining dishes for the dinner menu, whether one meat dish or a soup dish having the fourth label has been included in the first set of dishes may be first determined. A meat dish may comprise meat only or a mixture of meat and vegetables. The meat dish may be, for example, Peking duck, etc. The soup dish may be, for example, tomato and egg soup, spinach and egg soup, etc. If no meat dish or soup dish having the fourth label for the dinner menu has been included in the first set of dishes, one meat dish or one soup dish having the fourth label may further be randomly determined for the dinner menu from the third predetermined database to be included in the second set of dishes, so that to meet the determined target value for each intake category for the dinner menu and the at least one general rule as defined in step 106. If the actual value of calories for the one meat dish or one soup dish having the fourth label is larger than the determined target value of calories in the dinner menu, the amount (e.g., weight) of the one meat dish or one soup dish having the fourth label may be adjusted until the actual value of calories for the one meat dish or one soup dish having the fourth label is smaller than the determined target value of calories for the dinner menu. If one meat dish or one soup dish having the fourth label for the dinner menu has been included in the first set of dishes, the actual value of calories for the one meat dish or one soup dish having the fourth label may be calculated. If the actual value of calories for the one meat dish or one soup dish having the fourth label is smaller than the determined target value of calories for the dinner menu, whether one vegetable dish having the fourth label has been included in the first set of dishes may be determined as follows. A vegetable dish may only comprise vegetables.

If no vegetable dish having the fourth label for the dinner menu has been included in the first set of dishes, one vegetable dish having the fourth label may further be randomly determined for the dinner menu from the third predetermined database to be included in the second set of dishes, so that to meet the determined target value for each intake category for the dinner menu and the at least one general rule as defined in step 106. If one vegetable dish having the fourth label for the dinner menu has been included in the first set of dishes, a sum of the actual values of calories of the one meat dish or the one soup dish and the one vegetable dish having the fourth label may be calculated. If the sum of the actual values of calories of the one meat dish or the one soup dish and the one vegetable dish having the fourth label is larger than the determined target value of calories for the dinner menu, the amount (e.g., weight) of the one meat dish or the one soup dish and the one vegetable dish having the fourth label may be adjusted until the sum of the actual values of calories of the one meat dish or the one soup dish and the one vegetable dish is smaller than the determined target value of calories for the dinner menu. If the sum of the actual values of calories for the one meat dish or the one soup dish and the one vegetable dish having the fourth label is smaller than the determined target value of calories for the dinner menu, whether one staple dish having the fourth label has been included in the first set of dishes may be determined as follows.

If no staple dish having the fourth label for the dinner menu has been included in the first set of dishes, one staple dish having the fourth label may further be randomly determined for the dinner menu from the third predetermined database to be included in the second set of dishes, so that to meet the determined target value for each intake category for the dinner menu and the at least one general rule as defined in step 106. If one staple dish having the fourth label for the dinner menu has been included in the first set of dishes, the amount (e.g., weight) of the one staple dish having the fourth label is determined based on the actual values of calories of the one meat dish or the one soup dish and the one vegetable dish that have the fourth label and the determined target value for each intake category for the dinner menu, for example, the amount (e.g., weight) of the one staple dish having the fourth label is determined based on the intake difference between the sum of the actual values of calories of the one meat dish or the one soup dish and the one vegetable dish that have the fourth label and the determined target value for each intake category for the dinner menu and the at least one general rule as defined in step 106.

After determining the dishes including one staple dish, one meat dish or one soup dish, and one vegetable dish for the dinner menu, the dishes may be added to the meal plan as the dinner menu and may be recommended to the user.

In the provided method in the present invention, in case that the actual intake of one ingredient included in a meal history is greater than or equal to a predefined threshold, sending the user a notification and/or generating the meal plan without a dish containing the one ingredient based on the at least one general rule, wherein the meal history is a daily history or a weekly history, so that the user can have more balanced meal plans. For example, if the user had pork for five times in the past week, a notification that too much pork has been taken may be sent to the user, and/or the dish(s) containing pork in the generated meal plan may be replaced with dish(s) containing beef or lamb or fish.

The present invention may further comprise an apparatus comprising at least one processor, wherein the at least one processor may be configured to perform a part of or all steps in the present invention.

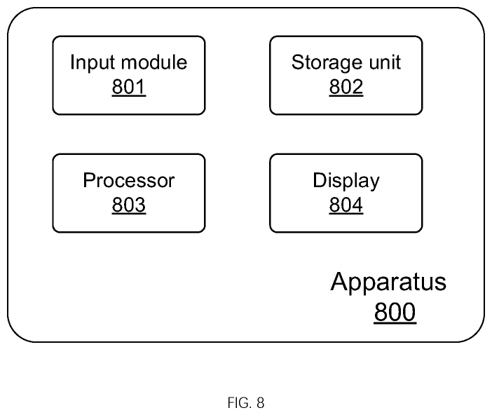

FIG. 8 shows a block diagram of an example apparatus according to the embodiment of the present invention.

The apparatus 800 may be configured to perform a part of or all steps in the present invention and may comprise an input module 801, a storage unit 802, a processor 803, and a display 804. The electronic device 800 may be a mobile phone, a tablet, a laptop, etc.

The input module 801 may be used to input information by a user. The information may be at least one of diet preference information, existing ingredient information, user information, etc. For example, the input module 801 may be a keyboard, a microphone, a touch screen, a camera, etc.

The storage unit 802 may store the information that is needed in the method in the present invention (e.g., in the methods in FIGS. 1-7). For example, the storage unit 802 may store at least one of diet preference information, existing ingredient information, user information, at least one general rule, label information, meal history, etc., that is input by the user or is predetermined, and programs and/or instructions for determining a first set of dishes from a predetermined dish set, determining a target value for each intake category, determining an actual value for the each intake category in the first set of dishes, determining a second set of dishes from the predetermined dish set, and generating a meal plan. Optionally, the storage unit 802 may further store programs and/or instructions for categorising at least one dish in the first set of dishes into an extra meal menu, a breakfast menu, a lunch menu, and a dinner menu depending on the label information, and programs and/or instructions for sending the user a notification and/or generating a meal plan in case that the actual intake of one ingredient included in a meal history is greater than or equal to a predefined threshold.

The storage unit 802 may include a volatile memory, a non-volatile memory, or a combination of a volatile memory and a non-volatile memory. The storage unit 802 may be provided in the apparatus. Alternatively or additionally, the storage unit 802 may be provided on a server connected via a network such as the Internet (e.g. cloud storage). The storage unit 802 may be a cumulative storage, where data is added with each new input. The storage unit 802 thus may include all input data.

The processor 803 may be configured to perform a part of or all steps in the present invention (e.g., the methods in FIGS. 1-7). For example, the processor 803 may be configured to determine a first set of dishes from a predetermined dish set, determine a target value for each intake category, determine an actual value for the each intake category in the first set of dishes, determine a second set of dishes from the predetermined dish set, and generate a meal plan. Optionally, the processor 803 may be further configured to categorise at least one dish in the first set of dishes into an extra meal menu, a breakfast menu, a lunch menu, and a dinner menu depending on the corresponding label, and may be further configured to send the user a notification and/or generate a meal plan in case that the actual intake of one ingredient included in a meal history is greater than or equal to a predefined threshold.

The display 804 may be configured to display, for example, at least one of the information input by the user, the information determined by the processor, and the information generated by the apparatus, in the methods in the present invention.

The input module 801 and the display 804 may be separate components of the electronic device. The input module 801 and the display 804 may be integrated. For example, the input module 801 may be part of the display 804.

The present invention may further relate to a storage medium comprising program instructions to instruct an electronic device to perform any of the methods mentioned above.

Meanwhile, while the specific embodiment has been described in the explanations of the present disclosure, it will be noted that various changes may be made therein without departing from the scope of the disclosure. Thus, the scope of the disclosure is not limited and defined by the described embodiment and is defined the scope of the claims.

1. A method for generating a meal plan, comprising:
obtaining diet preference information of a user;
obtaining existing ingredient information;
determining a first set of dishes from a predetermined dish set based on the existing ingredient information and the diet preference information of the user;
obtaining user information;
determining a target value for each intake category based on the user information;
obtaining at least one general rule;
determining, in the first set of dishes, an actual value for the each intake category;
determining a second set of dishes from the predetermined dish set based on differences between the target values and the actual values in the first set of dishes and the at least one general rule,
generating a meal plan comprising the first set of dishes and the second set of dishes.

2. The method of claim 1, wherein the diet preference information comprises at least one of taste preference, season preference, cooking type preference, and cuisine preference.

3. The method according to any one of the preceding claims, wherein the existing ingredient information comprises information of one or more ingredients, and the existing ingredient information is obtained via text input of the user, or recognized from an image, or selection of the user from a predetermined database.

4. The method according to any one of the preceding claims, wherein each of the first set of dishes comprises at least one of the one, two or more ingredients comprised in the existing ingredient information.

5. The method according to any one of the preceding claims, wherein the predetermined dish set comprises a set of dishes stored in a predetermined database, and the predetermined database further comprises information on at least one food category and predetermined dish packages.

6. The method of claim 5, wherein each of the predetermined dish packages comprises at least two dishes, wherein the at least two dishes are included in the meal plan in case that one of the at least two dishes is determined in the first set of dishes.

7. The method of claim 5 or 6, wherein the at least one food category comprises at least one of grains and cereals, fruit, dairy products, nuts, egg, meat, and vegetable.

8. The method according to any one of the preceding claims, wherein the user information comprises at least one of allergens, restricted ingredients, gene/DNA information, a number of meals for a day, height, current weight, expected weight, gender, active level and/or occupational information, location information, diet history data, user pattern data, family history information, and/or stage of the user, wherein the stage of the user is one of pre-pregnancy, pregnancy, puerperium, or lactation.

9. The method according to any one of the preceding claims, wherein the intake category is one of total calories, macronutrient, micronutrient, and at least one food category.

10. The method according to any one of the preceding claims, wherein the meal plan comprises at least one of a breakfast menu, a lunch menu, a dinner menu, and an extra meal menu, wherein the extra meal menu comprises at least one snack for at least one of the breakfast menu, the lunch menu, and the dinner menu.

11. The method of claim 10, wherein the at least one snack comprises at least one snack of dairy products, nuts, and fruit.

12. The method according to any one of the preceding claims, wherein the at least one general rule comprises at least one predetermined rule on energy distribution for a day, weight of each food category for a day, numbers of dishes for at least one of the breakfast menu, the lunch menu, the dinner menu, and the extra meal menu, and/or dish categories for at least one of the breakfast menu, the lunch menu, and the dinner menu, wherein the dish categories comprise staple dish, soup dish, meat dish, and vegetable dish.

13. The method of claim 12, wherein the predetermined rule on the energy distribution comprises:
the actual value of calories of the breakfast menu and the at least one snack for the breakfast menu is 30 percentage of the target value of total calories;
the actual value of calories of the lunch menu and the at least one snack for the lunch menu is 40 percentage of the target value of the total calories; and
the actual value of calories of the dinner menu and the at least one snack for the dinner menu is 30 percentage of the target value of the total calories.

14. The method of claim 12 or 13, wherein the weight of each food category is obtained based on the user information, preferably based on the stage of the user, wherein the stage of the user is one of pre-pregnancy, pregnancy, puerperium, or lactation.

15. The method according to any one of claims 10-14, wherein the breakfast menu comprises three dishes.

16. The method according to any one of claims 10-15, wherein the lunch menu comprises three dishes.

17. The method according to any one of claims 12-16, wherein the dish categories for the lunch menu comprises a staple dish, a soup dish, a meat dish or a vegetable dish.

18. The method according to any one of claims 10-17, wherein the dinner menu comprises three dishes.

19. The method according to any one of claims 12-18, wherein the dish categories for the dinner menu comprises a staple dish, a soup dish or a meat dish, and a vegetable dish.

20. The method of claim 12, wherein the extra meal menu comprises two snacks for the breakfast menu, two snacks for the lunch menu, and one snack for the dinner menu.

21. The method of claim 12, wherein the dish categories for the extra meal menu comprises nuts, dairy products, and fruit.

22. The method according to any one of the preceding claims, after the determining of the first set of dishes from the predetermined dish set, the method further comprises:
in case that at least one dish in the first set of dishes has a first label, categorising the at least one dish having the first label into an extra meal menu;
in case that at least one dish in the first set of dishes has a second label, categorising the at least one dish having the second label into a breakfast menu;
in case that at least one dish in the first set of dishes has a third label, categorising the at least one dish having the third label into a lunch menu; and
in case that at least one dish in the first set of dishes has a fourth label, categorising the at least one dish having the fourth label into a dinner menu.

23. The method of claim 22, wherein the determining of the second set of dishes from the predetermined dish set comprises:
determining the target value for the each intake category in the extra meal menu based on the at least one general rule, and determining one or more dishes having the first label from the predetermined dish set for the extra meal menu based on the at least one general rule in case that the actual value for the each intake category in the first set of dishes categorised into the extra meal menu is smaller than the target value for the corresponding each intake category in the extra meal menu;
determining the target value for the each intake category in the breakfast menu based on the at least one general rule, and determining one or more dishes having the second label from the predetermined dish set for the breakfast menu based on the at least one general rule in case that the actual value for the each intake category in the first set of dishes categorised into the breakfast menu is smaller than the target value for the corresponding each intake category in the breakfast menu;
determining the target value for the each intake category in the lunch menu based on the at least one general rule, and determining one or more dishes having the third label from the predetermined dish set for the lunch menu based on the at least one general rule in case that the actual value for the each intake category in the first set of dishes categorised into the lunch menu is smaller than the target value for the corresponding each intake category in the lunch menu; and
determining the target value for the each intake category in the dinner menu based on the at least one general rule, and determining one or more dishes having the fourth label from the predetermined dish set for the dinner menu based on the at least one general rule in case that the actual value for the each intake category in the first set of dishes categorised into the dinner menu is smaller than the target value for the corresponding each intake category the dinner menu.

24. The method according to any one of the preceding claims, wherein the method further comprises:
in case that the actual intake of one ingredient included in a meal history is greater than or equal to a predefined threshold, sending the user a notification and/or generating the meal plan without a dish containing the one ingredient based on the at least one general rule, wherein the meal history is a daily history or a weekly history.

25. The method according to any one of the preceding claims, wherein the diet preference information is pre-stored or is obtained from a input module comprising a voice recognition module and/or a text recognition module.

26. The method according to any one of the preceding claims, wherein the user information is pre-stored or is obtained from a input module comprising a voice recognition module and/or a text recognition module.

27. The method according to any one of the preceding claims, wherein the determining of the target value is further based on standard nutrient recommendations according to the user information of the user.

28. The method according to any one of the preceding claims, wherein the determining of the target value is further based on biometric information, microbiome information, and/or blood glucose information of the user.

29. An apparatus comprising at least one processor, wherein the at least one processor is configured to perform any of claims 1 to 28.

30. A storage medium storing computer instructions, where the instructions are configured to control at least one processor to perform any of claims 1 to 28.
